(19) United States
(12) Patent Application Publication
(10) Pub. No.: US 2003/0153024 A1
(43) Pub. Date: Aug. 14, 2003

(54) Charged bio-molecule/binding agent conjugate for biological capture

(76) Inventors: Brian M. Sullivan, Manhattan Beach, CA (US); Denes L. Zsolnay, Rolling Hills Estates, CA (US)

Correspondence Address: PATENT COUNSEL, TRW INC., S & E LAW DEPT., ONE SPACE PARK, BLDG. E2/6051, REDONDO BEACH, CA, 90278, US

(21) Appl. No.: 10/374,828

(22) Filed: Feb. 25, 2003

Related U.S. Application Data
(63) Continuation-in-part of application No. 09/837,946, filed on Apr. 19, 2001, now Pat. No. 6,562,209.

(51) Int. Cl. G01N 33/53; G01N 33/537; G01N 33/543; G06F 19/00; G01N 33/48; G01N 33/50

(52) U.S. Cl. 435/7.92; 205/777.5; 702/19

(57) ABSTRACT

ELISA (and ELISA-like) procedures for detection of agents, such as bioagents, proteins and nucleic acids, incorporate electrically charged (3) recognition molecules (5) that bind to the specific agent (7) being sought, deposit the charged recognition molecules and bound suspect agent in a fluid, and incorporate an electric field (E) to move and/or position those charged molecules to specific locations within the solution during the immunoassay procedure.

### REFERENCE TO PRIOR APPLICATIONS

Reference is made to U.S. application, Ser. No. 09/837,946, filed Apr. 19, 2001, entitled “Automated Computer Controlled Reporter Device for Conducting Immunoassay and Molecular Biology Procedures,” of which the present invention is a continuation in part. Applicant claims the benefit of 35 U.S.C. §120 based on the foregoing application.

### FIELD OF THE INVENTION

This invention relates to automated reporter systems that control the movement and position of recognition molecules in the performance of immunoassays of bioagents and other molecular biology test procedures used to detect bioagents and to make diagnoses, and, more particularly, to a method and apparatus for controlling the movement and positioning of recognition molecules, such as antibodies, suspended in fluid, used in those reporter systems.

### BACKGROUND

Controlling the motion of a recognition molecule in process apparatus for conducting immunoassay and other molecular biology test procedures, referred to herein as automated reporter systems, and otherwise is not new. For one, that controlled motion is accomplished in a prior apparatus, created by the present inventors, that is described in U.S. application, Ser. No. 09/837,946, filed Apr. 19, 2001, entitled “Automated Computer Controlled Reporter Device for Conducting Immunoassay and Molecular Biology Procedures.” An embodiment of the automated apparatus disclosed in the cited application detects specific bioagents using an enzyme linked immunoassay, referred to as an “ELISA” process. That immunoassay requires a recognition molecule, such as an appropriate antibody, that attaches to (i.e. adsorbs) a suspect bioagent molecule. To control the position of the recognition molecule in a liquid solution during that ELISA process, the molecule is coated on a magnetic bead, which serves as a carrier for the recognition molecule in the solution. A permanent magnet in the apparatus produces a magnetic field that is used to move and position the coated magnetic bead within the vessel that holds the liquid solution, and, hence, the recognition molecule, to locations required during the performance of the immunoassay procedure.

The ELISA process constitutes an identification process that uses molecular interactions to uniquely identify target substances. A basic definition of ELISA is a quantitative in vitro test for an antibody or antigen (e.g., a bioagent) in which the test material is adsorbed on a surface and exposed to a complex of an enzyme linked to an antibody specific for the substance being tested for with a positive result indicated by a treatment yielding a color in proportion to the amount of antigen or antibody in the test material. Color and color intensity serves as the reporter or indicia of the antigen or antibody. The basic ELISA procedure is described more specifically, for one, in a book entitled Methods in Molecular Biology Vol. 42, John R. Crowther, Humana Press, 1995.

The “antibody specific for the substance being tested for” in the foregoing definition constitutes a recognition molecule, a molecule that is capable of binding to either reactant or product molecules in a structure-restricted manner. That is, the recognition molecule binds to a specific three-dimensional structure of a molecule or to a two-dimensional surface that is electrically charged and/or hydrophobic in a specific surface pattern. It may also be recognized that the same essential approach, referred to herein as ELISA-like, can also be used using other recognition molecules, such as aptamers, DNA (e.g., deoxyribonnucleic acid), RNA and molecular imprint polymers.

More recently, the foregoing definition of ELISA has been expanded beyond the colormetric approach to include a voltametric or amperiometric approach to detection and assay. In the latter, the rate of change of voltage or current conductivity is proportional to the amount of antigen or antibody contained in the test sample. Such an approach is found in published PCT application PCT/US98/16714, filed Aug. 12,1998 (International Publication No. WO 99/07870), “Electrochemical Reporter System for Detecting Analytical Immunoassay and Molecular Biology Procedures” (hereafter the “16714 PCT application”). That published application describes both a colormetric and an electrochemical reporter system for detecting and quantifying enzymes and other bioagents in analytical and clinical applications. The electrochemical reporter system of the 16714 PCT application employs a sensor for detecting voltametric and/or amperiometric signals that are produced in proportion to the concentration of organic (or inorganic) reporter molecules by redox (e.g., reduction-oxidation) recycling at that sensor.

In brief, in the run of the ELISA process, the suspect bioagent is initially placed in a water-based buffer, such as a phosphate buffered saline solution, to form a sample solution. That sample solution is mixed with a quantity of particles, such as beads, coated with an antibody to the suspect bioagent (e.g., the recognition molecule, also sometimes referred to as a receptor molecule). The particular antibody used to coat the beads is one that is known to bind to the bioagent of interest (e.g., the target molecule) and is a primary antibody or “1° Ab.” Binding is the chemical “stickiness” that is selective to specific bioagents.

Any bioagent that is present in the sample solution binds with a non-covalent bond to a respective recognition molecule (e.g., antibody) and thereby becomes attached through the recognition molecule to a respective one of the magnetic beads in the sample solution. If the sample solution does not contain a bioagent or if the bioagent that is present in the solution is not one that binds to the selected antibody, then the recognition molecule on the bead remains unbound or free. Continued processing of the ELISA process in that case reports nothing. However, if the suspect bioagent is present in the sample solution, the bioagent binds to the recognition molecule coated on the beads. What then results is a quantity of bioagent molecules indirectly bound (through the recognition molecule), respectively, to a like quantity of coated beads. The mixture is optionally washed, as example, in a phosphate-buffered saline, and a second antibody, more specifically, an antibody and enzyme linked combination, is then added to the mixture.

The second antibody is also one that is known to bind to the suspect bioagent, and constitutes another recognition molecule, which may, but need not, be identical to the first antibody. The second antibody may either be one that is monoclonal, e.g., one that binds to only one specific molecule, or polyclonal, e.g., a mixture of different antibodies each of which shares the characteristic of bonding to the target bioagent. The enzyme is covalently bound to the second antibody and forms a complex that is referred to as a secondary antibody-enzyme conjugate or “2° Ab-enz.” As known by those skilled in the art, an enzyme is a “molecular scissors,” a protein that catalyzes a biological reaction, a reaction that does not occur appreciably in the absence of the enzyme. The enzyme is selected to allow the subsequent production of an electrochemically active reporter.

The2° Ab-enz binds to the exposed surface of the immobilized bioagent to form an “antibody sandwich” with the bioagent forming the middle layer of that sandwich. The antibody sandwich coated beads are washed again to remove any excess 2° Ab-enz in the solution that remains unbound.

The magnetic beads and the attached antibody sandwiches, the 1° Ab/bioagent/2° Ab-enz complex, in the solution are placed over the exposed surface of the redox recycling sensor. The substrate of the foregoing enzyme is added to the solution and the substrate is cleaved by the enzyme to produce an electrochemically active reporter. The substrate of the enzyme, referred to as PAP-Docket GP, is any substance that reacts with an enzyme to modify the substrate. The effect of the enzyme is to separate or cut the PAP, a para-amino phenol, the electrochemically active reporter, from the GP, an electrochemically inactive substance.

The foregoing chemical reaction is concentrated at the surface of the sensor. The rate of production of the foregoing reporter (e.g., the PAP) is proportional to the initial concentration of bioagent. The reporter reacts at the surface of the sensor, producing an electrical current through the sensor that varies with time and is proportional to the concentration of the bioagent, referred to as redox recycling. The occurrence of the electric current constitutes a positive indication of the presence of the suspect bioagent in the sample. Analysis of the electric currents produced over an interval of time and comparison of the values of that electric current with existing laboratory standards of known bioagents allows quantification of the concentration of bioagent present in the initial sample.

The automated apparatus of the '946 application, hereafter sometimes referred to as the automated ELISA system, provides a user friendly stand-alone portable system that automatically performs the ELISA process. The automated ELISA system contains a number of solutions in respective reservoirs and pumps that are controlled by a programmed computer. The electronic controller, such as a programmed microcontroller, controls a series of electric pumps to automatically sequence the pumping of the individual solutions required by the ELISA procedure into and out of a cell (or cells) as required by the ELISA program. That automated ELISA system uses coated beads of magnetic material and a magnetic positioning device to manipulate and position the coated magnetic beads under control of the computer, such as during the washing steps of the ELISA process, and in positioning the beads at the sensor during the redox recycling that yields the PAP. The controller commands the steps necessary to produce the reporter, controls the positioning of the carrier of the reporter adjacent the reporter sensor, analyzes the data obtained from the reporter sensor and displays the concentration of the bioagent determined from the analysis of the foregoing data. Once started, the apparatus, governed by the program, conducts the test automatically without the necessity for human intervention.

In a first step of the assay procedure, the sample solution, containing the sample that is to be tested for the presence of a specific bioagent, is placed in a container (or equivalent vessel) that holds the 1° antibody coated magnetic beads. If the sample is of the specific bioagent, then the respective parts of the sample links, sticks to, the antibody coating of a respective bead.

For example, the sample solution that is to be tested for the presence of a specific bioagent and the coated magnetic beads are pumped from respective reservoirs into a container by electrical pumps and mixed to ensure that the respective parts, that is, molecules, of the sample contacts the coating of a respective bead. In practice, the volume of the sample and bead solution is small and the container, which may be a length of pipette tubing, is also small. A known practical way to mix the ingredients of the solution is to create turbulence by repeatedly pumping the foregoing solution out of the container and then pumping that solution back into the container or to recirculate that solution.

Some of the sample may be unattached to a bead and that excess needs to be removed from the solution by washing. To wash the mixture, the magnetic beads (and attached molecules) are pulled by a magnetic field controlled by the controller to one side of the container, vacating a portion of the solution. An aspirating pipe is immersed in that vacated portion of the solution. The controller causes pumps to remove the dirty solution through the aspirating pipe and to replace the dirty solution with clean solution, thereby washing the magnetic beads.

The magnetic beads are preferably micron sized. Collectively, the quantity of micron sized magnetic beads in the solution appear as sludge; like mud in appearance and consistency, and is abrasive. Such sludge like collection of beads is known to be exceptionally hard on the valves and pumps of the automated apparatus, prematurely reducing the operational life of those valves and pumps. From the standpoint of improving the operational life of the valves and pumps, the elimination of the abrasive magnetic beads is clearly desirable. However, without those magnetic beads, the automated apparatus could not function. Until the present invention, no way of eliminating the magnetic beads was known. As an advantage, the present invention provides an automated apparatus that eliminates the magnetic beads yet controls the movement of the recognition molecules. The operational life of the pumps and valves in the automated apparatus is enhanced.

Accordingly, a principal object of the present invention is to increase the operational life of the pumps and valves of the automated ELISA apparatus.

An additional object of the invention is to eliminate the magnetic beads from the automated ELISA apparatus while retaining the ability of the apparatus to control the movement and positioning of the recognition molecules during operation of the apparatus.

A further object of the invention is to permit an applied electric field to move and position the recognition molecules.

And a still further object of the invention is to compound or otherwise create or build recognition molecules that are electrically charged.

### SUMMARY OF THE INVENTION

In accordance with the foregoing objects, ELISA (and ELISA-like) procedures employs electrically charged recognition molecules which, by design, bind to a specific bioagent. Those bioagent bound charged recognition molecules are positioned within a confined solution by applying an electrostatic field (e.g., electric field) to the solution to attract those charged molecules to a desired location within the vessel confining that solution. In the reporting stage of the ELISA and ELISA-like procedure the bioagent bound charged recognition molecules are further bound to a recognition molecule linked to an enzyme to form the recognition molecule/bioagent/recognition molecule enzyme complex. Due to the electric charge on the first recognition molecule in that complex, the complex may be moved by the electric field to a position within the solution adjacent a reporter sensor. When the substrate of the enzyme, the PAP-GP molecules, is introduced into the solution, the enzyme cuts or releases the PAP molecules (e.g., the reporter molecules) from the PAP-GP molecule at a location adjacent the sensor. The sensor detects the reporter molecules, and, indirectly, detects the presence and concentration of the suspect bioagent molecules that are present in the solution through changes in resistivity of the solution produced by those reporter molecules.

In accordance with a specific aspect to the invention, recognition molecules are given a significant electrical charge by attaching a polyglutamate to each recognition molecule using standard molecular biological techniques, and those charged molecules are deposited in a buffer solution. The inherent electrical charge of the polyglutamates becomes an electrical charge associated with each recognition molecule and polyglutamate linked combination molecule.

The incorporation of the electrically charged recognition molecules within the automated apparatus provides additional benefit. The electrically charged recognition molecules are smaller and weigh less than the corresponding micron sized recognition molecule coated magnetic beads used in the prior automated ELISA apparatus. By eliminating the greater bulk of the magnetic beads, the pumps, valves and containers can be made smaller in size, permitting greater miniaturization of the automated apparatus. Thus, not only is the operational life of the pumps and valves enhanced by eliminating the inherently abrasive magnetic beads, but the automated apparatus is smaller and lighter in weight than the earlier apparatus. That miniaturization enhances the overall utility of the automated apparatus.

The foregoing and additional objects and advantages of the invention, together with the structure characteristic thereof, which were only briefly summarized in the foregoing passages, will become more apparent to those skilled in the art upon reading the detailed description of a preferred embodiment of the invention, which follows in this specification, taken together with the illustrations thereof presented in the accompanying drawings.

### BRIEF DESCRIPTION OF THE DRAWINGS

In the drawings:

FIG. 1 is a pictorial illustration of the principal steps carried out by the principal embodiment of the invention;

FIG. 2 is a pictorial illustration of the electrostatic field structure used in the process of FIGS. 1 and FIGS. 3-5;

FIG. 3 is a block diagram of an embodiment of the invention;

FIG. 4 is a block diagram of a second embodiment of the invention; and

FIG. 5 is a partial schematic diagram of a third embodiment of the invention that improves upon the embodiment of FIG. 4.

### DETAILED DESCRIPTION OF THE PREFERRED EMBODIMENTS

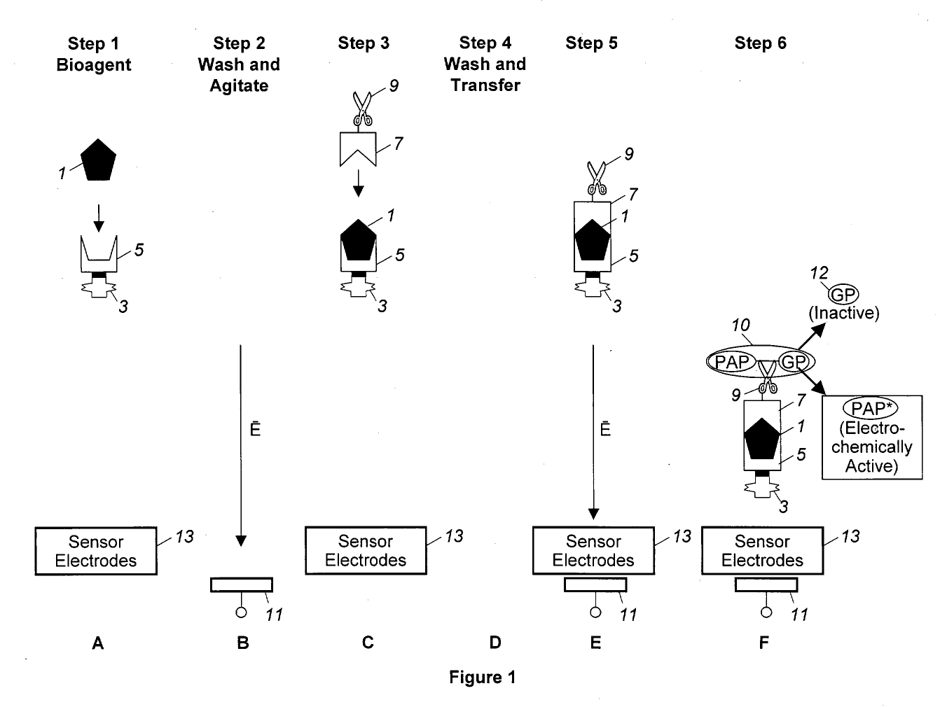

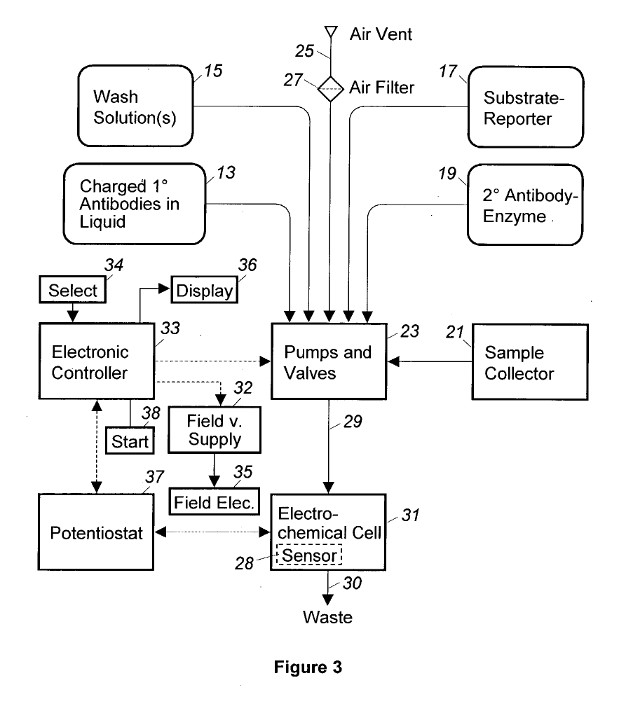

FIG. 1, to which reference is made, pictorially illustrates the various steps of an automated ELISA process that incorporates the improvements of the present invention. The description of this figure introduces the principles involved in this new process and apparatus. With an understanding of the general description accompanying this FIG. 1, the structure of the embodiments of FIGS. 3 through 5, later described, should be more readily understood and the operation more easily followed. In FIG. 1 STEPS 1 through 6 of the process are arranged in columns, left to right.

In preparation for the testing, a sample solution is produced by placing the suspect material, bioagent 1, represented in the figure by the pentagonal shaped symbol, in a water-based buffer, such as a phosphate buffered saline solution. The foregoing sample solution is to be tested for the presence of a specific bioagent or may be a solution that is simply suspected of containing a certain bioagent. If desired, the suspect material may be preliminarily treated, such as by exposing the material to ultrasonic energy, which breaks the material into multiple small clumps or even granules, which ensures maximum surface area exposure of the sample when placed in solution.

As further preparation, a supply of electrically charged recognition molecules is produced using known molecular biological techniques and then placed in a solution. Recognition molecules 5 are known to bind to the bioagents of interest (or of concern) on contact with those bioagent molecules. That is, the recognition molecule exhibits a chemical “stickiness” that is selective to specific bioagents. The recognition molecule may be a single-chain variable fragment (“sFv”) molecule or monoclonal antibody of known DNA (e.g., nucleic acid) sequence and is functionally equivalent to a primary antibody or “1° Ab.” That known DNA sequence is one to produce a molecule that binds or attaches to a specific target molecule, such as bioagent 1. Such monoclonal antibodies are typically grown from the cells of a mouse using known molecular immunology techniques.

The extent of electrical charge inherently associated with a given protein varies and is slight. The website of the Nanogen company, www.nanogen.com, notes that biological molecules possess natural positive or negative charges and may be attracted by an area of opposite charge. That company purports to employ such natural charges in DNA to permit the DNA to be captured on the surface of a semiconductor chip containing appropriately placed electrical charges, and then be operated upon. However, the slight charge of proteins is insufficient for the immunoassay process described herein, and is not regarded herein as being electrically charged as that term is used herein. Hence, the recognition molecule is modified to increase the electrical charge associated with the molecule, either positively or negatively in charge, beyond the inherent charge. That increase in electrical charge is accomplished by splicing the DNA that codes for a polyglutamate onto the monoclonal antibody, such as described herein in the final paragraphs of this specification.

Glutamates are known to inherently possess a more robust electrical charge. If more than one glutamate is attached in series (e.g., a polyglutamate) the net electrical charge increases roughly proportionately. Thus, the DNA code of the ultimate biological “product,” the electrically charged recognition molecule, contains two DNA segments, each segment individually characterizing something different. In this circumstance, the segments respectively characterize the monoclonal antibody that serves as the recognition molecule and the polyglutamate. In simplified terms, the foregoing splicing process produces a recognition molecule 5 that may be referred to as containing a “tail,” represented by the lantern shaped symbol 3 in the figure, of electric charge. The level of that electric charge is a function of the number of glutamate molecules used to form that tail.

The electrically charged recognition molecule is deposited in a fluid, such as deionized water that is buffered with some salts, as example, sodium phosphate to control the pH of the solution and sodium chloride to control ionic strength. Due to the electrically charged character of the tails, the antibody 5 and tail 3 combination can be manipulated in the solution by an externally applied electric field.

Continuing with FIG. 1, in the first step of the test, STEP 1, sample solution 1 containing the bioagent is placed into a container containing a quantity of the electrically charged recognition molecules 5, here antibodies, to the suspect bioagent, and mixed. The electrically charged tails 3 to those antibodies, in this example, contain a negative charge (relative to earth or ground).

Any bioagent molecule present in the solution binds to a respective antibody 5 and thereby becomes attached to a respective one of the electrically charged antibodies in the solution to form a 1° Ab/bioagent conjugate. The foregoing bond between the antibody and bioagent is recognized as a non-covalent bond. As becomes apparent, if no bioagent is present in the sample solution or if the bioagent that is present in the solution is not one that binds to the selected antibody 5, then antibody 5 remains unbound. The results obtained from further processing as described herein will then show nothing. For purposes of the description of operation, the sample solution being tested is presumed to carry bioagent 1.

Accordingly, bioagent 1 bonds to antibody 5, as represented in STEP 3 by the seating of the pentagonal shaped bioagent symbol inside the mating cavity of the antibody symbol. On conclusion of STEP 1, the solution contains a quantity of bioagent molecules 1 bound respectively to a like quantity of electrically charged antibodies 5.

In STEP 2 the mixture in solution of STEP 1 is optionally washed before proceeding with step 3. A suitable wash solution, as example is phosphate-buffered saline. Variants of the foregoing wash solution may include additional components, such as bovine serum albumin or detergents. To wash the mixture in solution, an electric field, E, is first established and applied to the mixture. That electric field is either produced external of the vessel or other containment region holding the solution and is directed from a field electrode through a side of the vessel. The foregoing requires that the vessel or other containment region be constructed of dielectric material through which static electric fields may effectively extend, such as glass, ceramic or nylon.

Alternatively, the electric field may be produced inside the vessel, in which case, the field electrode or electrodes are located inside the vessel or confinement region, immersed in the solution.

Assuming the former electrode structure, the electric field, E, produced by electrode 11 effectively penetrates the walls of the vessel and attracts the negatively charged antibodies to the side closest to the positive electrode, leaving the solution at the other side of the vessel relatively free of the charged antibodies. The side of the solution in the vessel vacated by the antibodies is then aspirated to remove the solution, suitably using an aspirating tube, without removing the charged antibodies. The removed solution is then replaced with clean solution. The renewed solution is then agitated to re-suspend the antibodies 5 in the wash solution. The foregoing agitation is preferably accomplished by pumping fluid into and out of the reaction area of the vessel.

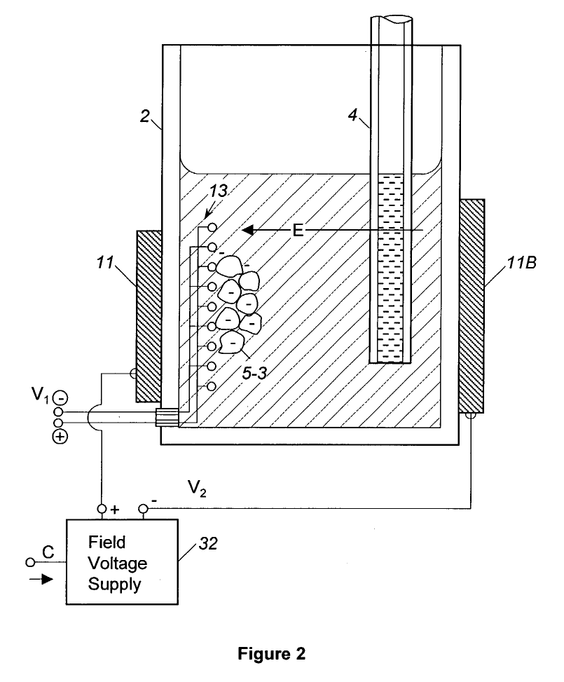

The structure for the foregoing operation is pictorially illustrated in FIG. 2 to which reference is made. Container 2 contains a fluid containing the bioagent and recognition molecule combination, aspirating tube 4, a sensor 13. The container also supports a pair of field electrodes 11 and 11B, located on diametrically opposite outer walls of the container. Sensor 13, comprising interdigitated electrodes, as example, is positioned along a wall of the vessel. Electrical leads 4 connected to the respective electrodes of the sensor extend through a wall for connection to the current monitoring apparatus, not illustrated, and to a voltage extended from that monitoring apparatus.

Field electrodes 11 and 11B are electrically connected to the opposite polarity terminals of a field voltage supply 32. The field voltage supply is ungrounded and electrically isolated from the voltage source, not illustrated, that is associated with the current monitoring apparatus, referred to previously.

Field voltage supply 32 is switchable between an “on” and “off” condition. When an appropriate switching signal or command is applied at input C of the field voltage supply, the supply generates the requisite voltage which appears between field electrodes 11 and 11B, with the polarity of electrode 11 being positive relative to electrode 11B. That voltage establishes the electric field E that extends from electrode 11B through the walls of container 2 and the solution, the opposite side of the container wall to electrode 11. The field is essentially a DC electrostatic field. The path between the electrodes is of very high resistance and virtually no current is drawn from field voltage supply unit 32. Typically, the voltage produced by the field voltage supply is under one volt, to avoid electrolysis of the water as could produce hydrogen and oxygen gases, a rather explosive combination. The negatively charged matter in the solution, charged antibody 5-3, is attracted to one side by the electric field, the left side in the figure, and vacates the opposite side, the right, of the container. The liquid of the solution may then be aspirated by a pump via aspirating tube 4, leaving the charged antibody alone; and the withdrawn liquid may be replenished by pumping clean liquid through that tube.

When the switching signal is removed or changed from the input C of the field voltage supply, the electric field voltage supply switches off and the electric field collapses, releasing the attractive force on the charged antibody combination and allowing that matter to circulate freely in the solution. As later herein described, in the automated system the field voltage supply 32 and the current monitoring apparatus associated with sensor 13 are controlled by a computer. Sensor 13 does not serve any function during the wash procedure, but performs a function in the later stages of the test process, later herein described.

Returning to FIG. 1, following the foregoing wash procedure of STEP 2, the electric field is again applied to again trap the charged antibodies and the wash process is repeated. The foregoing washing procedure is repeated as many times as experience shows is necessary to adequately clean the solution. As those skilled in the art appreciate, other washing protocols may be substituted for that described using the electric field without departing from the invention, but the electric field approach is believed most convenient.

In the next step, STEP 3, a second recognition molecule, more specifically, an antibody 7 and enzyme 9 linked combination-is added to the mixture in solution. The second antibody 7 is also one that is known to bind to bioagent 1 of interest. That antibody need not be the same structure as the first antibody, and may be either be one that is monoclonal, e.g., one that binds to only one specific molecule, or polyclonal, e.g., a mixture of different antibodies each of which shares the characteristic of bonding to the target bioagent. The enzyme 9, illustrated by the scissors symbol, is covalently bound to the second antibody 7 and forms a complex that is referred to as a secondary antibody-enzyme conjugate or “2° Abenz.”As is known, an enzyme is a “molecular scissors,” a protein that catalyzes a biological reaction, a reaction that does not occur appreciably in the absence of the enzyme. Enzyme 9 is selected so as to allow the subsequent production of an electrochemically active reporter, described hereafter in succeeding steps of the process.

The 2° Ab-enz conjugate binds to the exposed surface of the immobilized bioagent to form a charged 1° Ab/bioagent/2° Ab-enz complex, an “antibody sandwich.” The bioagent forms the middle layer of that sandwich, such as is illustrated in the pictorial of STEP 5 to which brief reference is made. Returning to completion of STEP 3, the next step, STEP 4, is to wash the charged antibody sandwich in a manner similar to, but not necessarily identical to that described in STEP 2. At this juncture in the test procedure, the purpose of the wash is principally to wash away any excess 2° Ab-enz that is not bound to a bioagent. If one employs a separate reaction chamber (as appears in one of the embodiments later described) and electrochemical cell instead of a single cell, following the foregoing washing, the charged 1° Ab/bioagent/2° Ab-enz complexes are transferred from such reaction chamber to the electrochemical cell prior to undertaking STEP 5.

In STEP 5, electrode 11 again produces electric field, E, such as is represented by the arrow in the illustration. The electric field is oriented to attract the electrically charged antibody sandwich, the 1° Ab/bioagent/2° Ab-enz complex, in the solution to the exposed surface of the sensor 13. That sensor may be the redox recycling sensor consisting of interdigitated electrodes, such as described in the published 16714 PCT application, earlier cited. Referring again to FIG. 2, at this stage, field voltage supply 32 is set “on” to generate the electric field. It is seen that the electric field E produced between electrodes 11 and 11b extends through the interdigital finger electrodes of sensor 13 and from those finger electrodes to electrode 11.

Next, in STEP 6 the substrate 10 of the enzyme is added to the solution and the substrate is cleaved by enzyme 9 to produce an electrochemically active reporter. The substrate of the enzyme is any substance that reacts with an enzyme to modify the substrate. This is illustrated in STEP 6 in which a preferred embodiment of substrate 10 is denominated PAP-GP. The effect of the enzyme is to cut the PAP, a para-amino phenol, the electrochemically active reporter, from the GP 12, an electrochemically inactive substance.

The electric field produced by electrode 11 concentrates the foregoing chemical reaction at the surface of the sensor. The rate of production of the foregoing reporter (PAP) is proportional to the initial concentration of bioagent. The reporter reacts at the surface of electrochemical sensor electrodes 13, producing an electrical current through the sensor electrodes that varies with time and is proportional to the concentration of the bioagent.

An analysis of the electric currents produced in the foregoing manner over an interval of time and comparison of the values of that current with existing laboratory standards of known bioagents allows quantification of the concentration of bioagent present in the initial sample. More specifically, a least-square linear regression analysis of the data generates the slope of the current, representing the rate of change of current over time. That slope is then compared with corresponding slopes that were previously obtained in measurements of standard concentrations of the bioagent. By selecting the closest match between the measured and reference slopes the amount of bioagent present in the initial sample is determined.

As those skilled in the art appreciate, the foregoing method (and apparatus) of FIGS. 1 and 2 modifies the prior automated ELISA procedure of the cited co-pending application Ser. No. 09/837,946, filed Apr. 19, 2001, for one, by incorporating into the process electrically charged antibodies that may be manipulated in position within a confined fluid and the electric field for controlling positioning of those charged molecules in lieu of magnetic beads and magnets. The foregoing process for performing the ELISA (and ELISA-like) sample analysis and a more detailed description of the apparatus used to carry out that analysis is presented in the embodiments illustrated in FIGS. 3-5, the description of which follows in this specification.

Reference is next made to FIG. 3, which illustrates in block diagram form a first practical embodiment of the automated system, referred to as a single stage apparatus. The apparatus includes four containers or, as variously termed, vessels 13, 15, 17 and 19, which hold, respectively, the electrically charged 1° antibody in a liquid solution, the wash solution, the substrate reporter and the 2° antibody-enzyme solutions earlier described. The apparatus further includes a region in which to collect the sample that is to be analyzed, such as an inlet region or a vessel. Each of the foregoing vessels is connected by appropriate fluid conduits, the plumbing, to the pumps and valves unit 23 of the apparatus as illustrated.

Pump and valve unit 23 houses individual pumps, not separately illustrated in the figure, for each of the respective vessels. An air vent 25 and air filter 27 are also plumbed into the pumps and valves unit. The air vent and air filter provides a vent to allow air to separate from solutions and/or remove solutions from tubes. A fluid conduit 29 extends from the pumps and valves unit into electrochemical cell 31, the vessel in which the examination is made. Waste conduit 30 extends from cell 31 to an appropriate sump or sewer, not illustrated, to permit disposal of the waste of the process. The apparatus includes an electronic controller 33, which is a programmed microprocessor or microcontroller, a field voltage supply 32, a field electrode 35 and a potentiostat 37, which are under control of the controller as represented by the dash lines. The potentiostat is electrically coupled to a current sensor 28, represented in dash lines, located inside cell 31. That current sensor is preferably formed of interdigitated electrodes, earlier described. Electronic controller 33 includes a selector 34 through which the operator may select the particular antigen for which the analysis of the sample is being undertaken, a start button 38 and a display 36, preferably a liquid crystal display (“LCD”), by which the assay may be reported to the operator.

Field electrode 35 is of metal. The field electrode is supported and positioned against examination cell 31 behind sensor 13. The field electrode is supplied with voltage when required by the field voltage supply 32. Normally, field voltage supply 32 does not apply voltage to field electrode 35. When voltage supply 32 receives the command from and is energized by the electronic controller, the field voltage supply generates the voltage that is applied to the field electrode, and that voltage in turn produces an electric field directed through the examination cell and through (and about) the electrodes of the sensor inside that cell. When a command is received from controller 33 to extinguish the electric field in examination cell 31, the field voltage supply is deenergized.

Potentiostat 37 supplies the voltage to the electrode array, the sensor 28, that monitors the reporter, described in step 4 of FIG. 1, earlier described disposed on the bottom or side of electrochemical cell 31. That is, the sensor carries any extra electrical current that flows in series through the electrode array and potentiostat as a result of the “redox recycling” reaction that takes place during the latter stage of analysis when the enzyme substrate is cleaved to release the reporter (Step 6 earlier described in FIG. 1). The potentiostat is also coupled to an input of the controller 33 and communicates the electrical current levels that flow through the interdigital array to the controller.

Electronic controller 33 is a programmed microprocessor, microcontroller, computer, as may be variously termed, or the like. The electronic controller controls each of the pumps and valves housed in unit 23 and controls energization of field voltage supply 32. The controller also enables and receives monitored current readings from potentiostat 37. Controllers of the foregoing type are quite small in size and may be housed or embedded in the structure of one of the units, such as in pumps and valves unit 23 so as to be inconspicuous. The foregoing components may all be packaged into a small size compact unit that may easily be carried by an individual. For added portability, the controller and pumps may be battery operated. Otherwise the apparatus may be supplied with electrical operating power from the facility in which used or by a motor generator set.

Electronic controller 33 includes a memory, not separately illustrated, such as ROM or EPROM to permanently store the operating system and the programs as well as temporary memory such as RAM, not separately illustrated. The principal programs of the controller are evident from the description of operation that follows. It will be realized that the controller serves as a sequencing device for controlling the pumps, as a collection point for data, and as a calculating machine for analyzing the data and displaying the result.

The electrochemical reaction sensor employed in the apparatus of FIG. 3 may be any type of sensor that supplies information on the reporter and supplies that information to the electronic controller. One such sensor applies a given voltage across at least two spaced electrodes disposed in the electrochemical cell and senses the level of electric current that flows between those electrodes. However the preferred sensor is of the interdigitated array type one that is described in the cited '16714 PCT application, IPN '870 application and publications cited in the background to this invention. The interdigitated array structure is promulgated as being the most sensitive and, hence, allows better resolution of the data than other known sensors to date in this application.

For operation, electrical power is connected to electronic controller 33. The operator determines the particular bioagent that is being sought in the sample material, preferably prepares the sample in accordance with the ultrasonic energy exposure earlier described, places the sample in a solution in sample collector 21, and selects the particular bioagent on selector 34. Vessels 13, 15, 17 and 19 are filled with the appropriate ingredients, earlier described and not here repeated. The operator operates the start button 38 and in response electronic controller 33 commences the automatic operation specified in the stored program.

The program of the controller motivates dispensing the contents of sample collector 21 into cell 31 by commanding the controller to briefly energize an electrical pump associated with the sample collector. The energized pump pumps the sample-in-liquid through the plumbing, including conduit 29, and into cell 31, which may be referred to as the examination cell. Concurrently or subsequently the program motivates dispensing of the contents of vessel 13, the electrically charged 1° antibodies in liquid, by commanding the controller to energize a second electric pump associated with that vessel for a short interval. The second electric pump pumps the electrically charged 1° antibodies into examination cell 31. Presuming the suspect bioagent is present in the sample material previously deposited in the cell, the bioagent binds to the antibody, as earlier described in STEP 1 of FIG. 1.

Although the description of the embodiment refers to individual pumps to accomplish the prescribed pumping, those skilled in the art recognize that other less expensive arrangements may be employed in alternative embodiments that accomplish the pumping with a configuration of electrically controlled valves and pumps that allows pumping of fluid from specific vessels as selected by the controller. For example, a valve could be associated with each vessel, the controller would select the particular valve to open, and then cause a pump to operate and draw the fluid through the valve.

Returning to the operation, following a short interval the controller program next commands the washing of the ingredients in the fluid in cell 31. For the washing operation, the program commands energization of field voltage supply 32, which supplies voltage to the electrode 35 to produce an electric field extending inside of examination cell 31, and commands energization of a third pump, not illustrated in the figure, referred to herein as the aspirating pump. The electric field draws the electrically charged antibodies to one side of the cell, vacating the charged antibodies from the solution on the other side of the cell. The aspirating pump connects to a conduit that extends into the vacated side of the solution, and the third pump aspirates the fluid and expresses the waste fluid through waste conduit 30. After a suitable interval the program halts the third pump and energizes a fourth pump that connects to a second vessel in wash solution 15 and pumps sufficient clean fluid to replace the fluid that was removed, completing the wash. The foregoing wash function corresponds to STEP 2 of FIG. 1. The solution is then agitated to re-suspend the charged antibodies in the solution as by aspirating a small amount of fluid from the vessel and then repumping the aspirated fluid back into the vessel often referred to as an “up-down” of the solution.

The foregoing washing procedure may be repeated the number of times required by the controller program, and the number written into the program is one that satisfies the requirements of a particular operator's experience. For purposes of this description, the washing step is performed once.

The program then motivates the delivery of the 2° antibody-enzyme into examination cell 31 by energizing the pump associated with vessel 19 for a predetermined interval. In the examination cell, the antibody-enzymes then bind to another region of the bioagent, producing the 1° Ab/bioagent/2° Ab-enz complex. The latter is the same as described in STEP 3 of FIG. 1.

The electric field produced by electrode 35 draws the electrically charged 1° Ab/bioagent/2° Ab-enz complex to sensor 28. This is the same as represented in STEP 5 of FIG. 1. The program of controller 33 next motivates the delivery of the substrate reporter in vessel 17 into the solution in examination cell 31 by commanding energization of an electric pump, not illustrated in the figure, associated with vessel 17. The pump is energized for a predetermined interval and pumps the substrate into the contents within examination cell 31. Cleavage of the substrate by the enzyme commences.

As recalled from the preceding paragraphs, the electric field that extends through the walls of electrochemical cell 31 draws the 1° Ab/bioagent/2° Ab-enz complex to the surface of the test electrodes of the electrochemical sensor, not illustrated in this figure, disposed inside cell 31. At that location adjacent the electrode surface of the sensor, the bound enzymes cleave the substrate to produce the reporter molecules.

Sensor 28 monitors the reaction and reports to the electronic controller 33. In turn, the controller program analyzes the data obtained. To monitor electric current through the examination cell the potentiostat applies a voltage across the spaced interdigitated electrodes, earlier described, which serve as sensor 28. That applied voltage produces an electrical current that passes from one spaced electrode, the anode, through the solution to the other electrode, the cathode. Absent a reaction in the solution, the electric current attains a certain default or base value, depending upon the resistivity of the solution. As the reaction commences to produce the reporter, the resistivity of the solution decreases, increasing the current. The effect is referred to by electrochemists as redox recycling. As the reaction continues producing greater numbers of reporter molecules, the resistivity changes further, as does the electric current. The rate of change of the current is a measure of the concentration of the selected bioagent. Information of the current, whether the information is in digital form or analog form, is coupled to electronic controller 33, which analyzes the changing data in real time.

Essentially concurrently with the pumping of vessel 17, the controller program commences the checking and assembling of the data on electrical current flow through the sensor by repetitively checking the current readings supplied by potentiostat 37 over a predefined interval of time. For example, one hundred readings may be taken equally spaced over an interval of ten minutes. The data obtained is temporarily stored in the memory of the electronic controller. The program then performs a least-square linear regression analysis of the data and the analysis generates the slope of the sensor current (e.g., change of current level vs. time), a number that represents the rate of change of current.

The electronic controller also stores in memory (ROM or EPROM) a library of the standards that have previously been established in the laboratory to identify bioagents or antigens and the concentration of the respective antigen in a solution by measuring the rate of change of current that occurs when using the known electrochemical ELISA procedure. Each antigen or bioagent produces a rate of change of current that depends on the concentration of the bioagent in the sample. For any given combination of recognition molecule(s) and bioagent or other antigen, a given concentration produces a unique rate of change of current. The increase in current as a function of time from the beginning of the chemical reaction to produce the reporter is essentially linear, and produces a straight line curve of the type I=at+b, where “t” represents time, “b” is an initial constant, a number, and “a” is the slope of the line, also a number. The foregoing slope information and the correlation of that information to respective concentration levels has been tabulated and serves as the standards.

Thus, for each combination of recognition molecule(s) and bioagent or other antigen that is to be studied, the library, often referred to as a “look-up table,” contains the correlation between the slope numbers and the concentration levels correlated to those slope numbers. After concluding the regression analysis and obtaining the slope number, the controller program checks to determine which bioagent or antigen was selected by the operator and then accesses the stored look-up table for the selected bioagent or antigen. The computer then compares the slope obtained in the foregoing regression analysis with corresponding slopes obtained in measurements of standard concentrations. Once the computer locates the closest match, the computer then displays the concentration of the antigen on display 36. Optionally, the computer may be programmed to also display the calculated slope. Further, since the volume of the electrochemical cell is known, the computer may also optionally display the total quantity of antigen in the test sample.

The foregoing apparatus is recognized as being automatic in operation, is very “user-friendly” and does not require highly skilled personnel to operate. Incorporated within a compact housing and with optional battery or house supply power the apparatus is portable and suited for use on location.

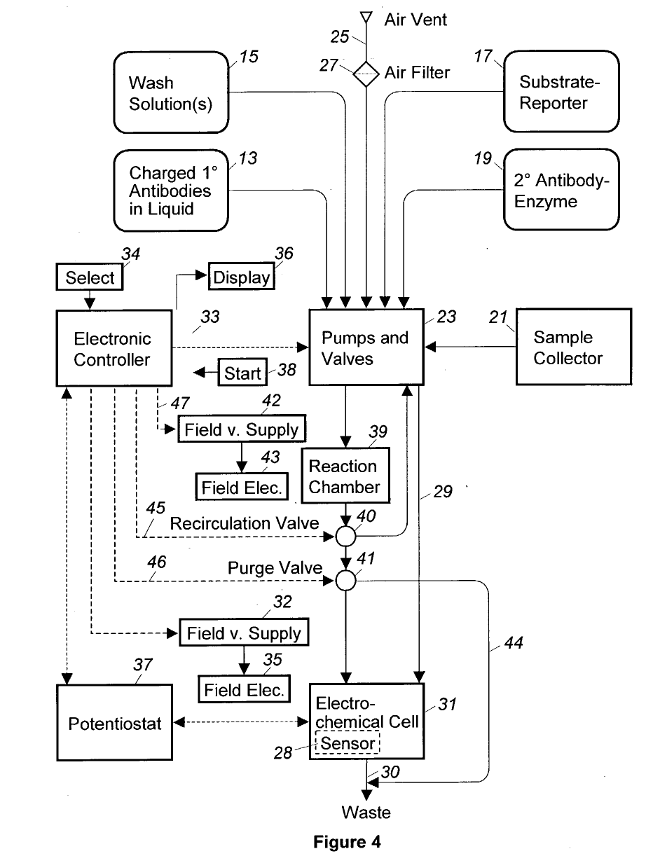

Reference is made to FIG. 4 that shows a block diagram of an alternative embodiment of the apparatus. This second embodiment is regarded as a two stage apparatus, whereas the apparatus of FIG. 1 is regarded as a single stage apparatus. For convenience, the components of the embodiment of FIG. 3 that are essentially the same in structure as those previously described in FIG. 1 are given an identical denomination. Those components that are changed slightly are denominated by the same number used for the corresponding element and the numbers are primed.

As inspection of FIG. 4 shows that many of the functional elements of this embodiment are the same as in the prior embodiment. The components that have been added include a separate reaction cell or chamber 39, recirculation valve 40, purge valve 41, an additional field voltage supply 42 and associated field electrode 43, some additional fluid conduits, some additional outputs and control lines from the electronic controller, and a slightly changed program for the electronic controller to accommodate the additional components and functions.

In this embodiment the reactions and washes are carried out in a separate vessel, the reaction chamber 39. A separate field voltage supply 42 and electric field electrode 43 are employed in connection with the dielectric walled reaction chamber. The plumbing and pump arrangement also differs. The electronic controller is programmed to handle the functions that correspond to steps 1-6 of FIG. 1 and all of the same operation as in the embodiment of FIG. 3, excepting the cleavage operation that generates the reporter. In the foregoing field voltage supply 42 and electric field electrode 43 are used the same as that described for field voltage supply 32 and field electrode 35 in the embodiment of FIG. 3.

At the reporting stage in the present embodiment, the controller opens valves 40 and 41 permitting the now electrically charged 1° Ab/bioagent/2° Ab-enz complex in solution to transfer from the reaction chamber 39 into examination cell 31, and commands field voltage supply 32 to apply the voltage to field electrode 35 to direct the electric field into cell 31 and through the sensor 28. The controller then directs the final chemical, substrate reporter 17, to be pumped via conduit 29 into the examination cell 31. As in the prior embodiment, electronic controller 33 senses the electrical current through the sensor and potentiostat 37, which is changing, determines the rate of change of current, e.g., the slope, and from that slope determines the concentration of the bioagent. The controller then displays the concentration on display 36. Upon conclusion of the examination the contents of the cell are expressed through conduit 30 as waste.

The embodiment of FIG. 4 includes some additional features. Valve 40 is referred to as a recirculation valve. Should the program call for recirculating the solution, the controller sets valve 40 to open a path into a circular conduit. An aspirating pump, not illustrated, located within unit 23 pumps the solution to mix the solution.

Valve 41 is referred to as the purge valve. Instead of commanding that the solution in chamber 39 be pumped into cell 31, the controller may instead set valves 40 and 41 to open a passage into conduit 44 and then initiate an electric pump that pumps the solution in chamber 39 through the valves and out conduit 44. Conduit 44 leads into conduit 30 and leads to the waste disposal system.

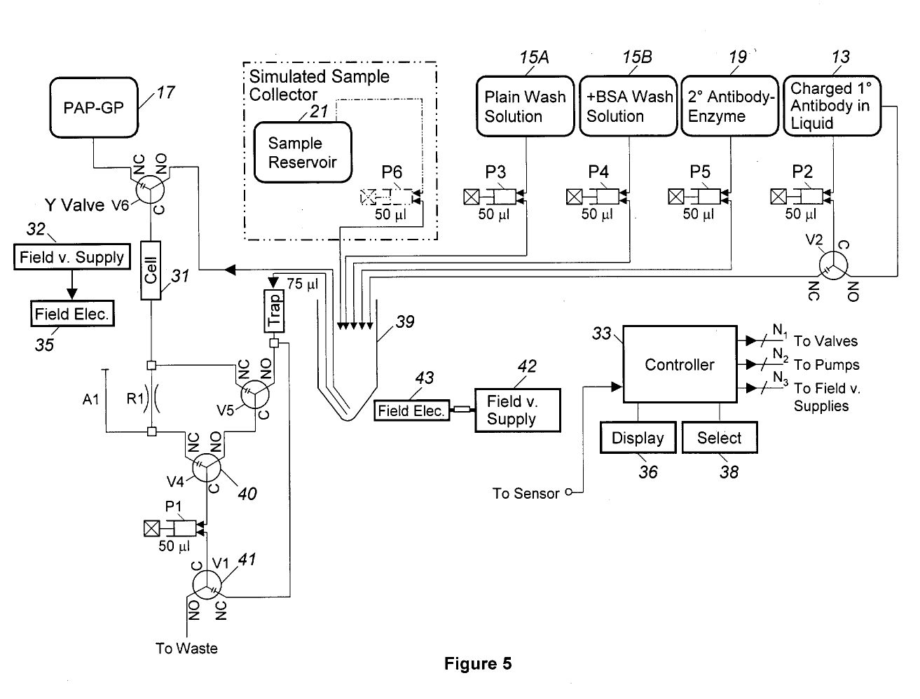

Reference is next made to the schematic illustration of FIG. 5, which illustrates another embodiment of the invention, a variation of the embodiment of FIG. 4, earlier described. For convenience, the denomination of the components in this embodiment, which are the same as those used in the embodiment of FIG. 4, are identified by the same number, with few exceptions. The embodiment of FIG. 5 contains electrically operated pumps P1, P2, P3, P4, P5 and P6 and a series of electrically operated valves, V2, Vl, V4, V5 and V6, all of which are controlled by the controller 33. The convention adopted to describe the condition of a valve when referring to same as either open or closed may be stated briefly. When a valve outlet (or inlet) is referred to as being “closed,” the term means that the outlet is blocked so that fluid cannot flow there through. When the valve outlet (or inlet) is said to be open, the term means that the outlet (or inlet) is unobstructed, and fluid is able to flow there through. Each of the foregoing valves is a two-way valve and contains an inlet and a pair of outlets, one of the outlets being normally closed, as illustrated by a gap, and the other of which is normally open, as represented by an unbroken line. When the valve is energized, the foregoing state of the outlets reverses.

Controller 33, display 36 and start switch 38 are illustrated in block form. The controller outputs to the respective valves, pumps and positioners are represented by cable outputs N1, N2 and N3, in which the cable contains the requisite number of electrical leads, N, for the respective components associated with the cable. To avoid undue complication to the schematic, the electrical leads are not extended to the respective controlled component in as much as those skilled in the art will understand the connections. Likewise the input lead from the sensor, not illustrated, disposed in examination cell 31, is only partially illustrated.

Pump P2 is associated with vessel 13 and is for pumping the electrically charged antibodies in liquid solution contained in the vessel through the valve V2 and, when the valve is energized, the plumbing lines into reaction cell 39. Valve V2 is a two-way valve. The valve contains a normally closed passage that leads into a conduit that in turn terminates in vessel 13, forming a recirculating fluid loop in the system. Thus, when pump P2 is energized by the controller, and valve V2 remains deenergized, such as illustrated in the figure, the charged antibody solution is pumped through the recirculating loop. The recirculation of the charged antibody solution helps to homogenize the distribution of the antibodies in the solution. When both pump P2 and valve V2 are energized, the valve opens the recirculating loop and closes the passage through the conduit into reaction cell 39.

Pumps P3 and P4 are associated with vessels 15A and 15B. The two vessels contain different wash solutions, as example, phosphate buffered saline solution in 15A and Bovine Serum Albumin (“BSA”), respectively. The BSA is a main component of cow blood in water, a random protein that prevents the charged antibodies from competing for binding sites. Thus instead of a single wash in this embodiment, a double wash with different washing solutions is accomplished. When commanded by the controller, pumps P3 and P4 will respectively pump the contents of vessels 15A and 15b through respective conduits into reaction cell 39.

Pump P5 is associated with vessel 19 containing the 2° antibody-enzyme and pumps the contents into the foregoing cell via a separate conduit into the reaction cell. Pump P6 is associated with vessel 21 in which the sample of bioagent is placed in liquid solution. The pump pumps the sample solution through a separate conduit into the reaction chamber.

The enzyme substrate (PAP-GP) is contained in vessel 17. Valve V6 contains a normally closed inlet, a normally open inlet and an outlet. The normally open inlet connects via a conduit to vessel 17 and opens in the bottom side of that vessel. The normally closed inlet connects via a conduit to an aspiration tube that is disposed in reaction cell 39. The outlet of the valve connects through a conduit to the upper end of the examination cell 31. The examination cell contains an outlet at the bottom end of the cell that connects via a conduit to a normally open inlet of Valve V4 and to a standpipe A1 that opens to the atmosphere. The foregoing conduit also includes a flow restrictor R1.

Each of valves V4 and V5 contain a normally open inlet, a normally closed inlet and an outlet. Valve V1 contains an inlet, a normally closed outlet and a normally open outlet. Pump P1 is connected by conduit in series between the outlet of valve V4 and the inlet of valve V1. The normally open outlet of valve V4 connects to the outlet of valve V5 and the normally open inlet of valve V5 connects via a trap and conduit to a second aspiration tube that extends into the reaction cell 39.

Assuming that the stage of operation of the foregoing system is ready to examine for the bioagent in examination cell 31, the solution located in examination cell 39 must be transferred into the examination cell 31 and the enzyme substrate (PAP-GP) must be added thereto. The transfer is accomplished by aspirating the solution from the examination cell by operating pump P1 and valve V4. In operating pump P1 creates an aspirating force inside cell 31 through the now closed inlet of valve V4 and the conduit into expelling gas and/or fluid through the inlet and normally open outlet of valve V1. The short closed fluid tube A1, referred to as an accumulator, is also connected in common with the normally closed passage in valve V4. The accumulator is filled with air and serves as an “air spring” that evens out the flow rate of the solution to a uniform slow fluid motion. The draw pulls solution from reaction cell 39 via the aspirating tube, the normally open inlet of Valve V6 and the outlet of that valve and into cell 31. The amount of time required to pump and adequately fill the examination cell is pre-calibrated during the design of the system and is known to the program in the controller.

When the foregoing transfer is completed, the controller then additionally energizes valve V6. With energization, the normally closed valve inlet of Valve V6 is switched to open (and vice-versa for the normally open valve inlet). Pump P1 aspirates a portion of the contents of cell 31 containing the sample while drawing the PAP-GP from vessel 17 through valve V6 and into cell 31.

The foregoing system operates essentially the same as previously described for the preceding embodiment. Prior to operating valve V6, the controller readies the examination cell for detection of the redox recycling that is expected to occur. Thus, controller 33 commands field voltage supply 32 to energize and apply the appropriate voltage to the electrode 35 and establish the appropriate electric field within examination cell 31 with that field passing through the electrochemical sensor, not illustrated in the figure, inside the cell. When the PAP-GP is subsequently introduced into cell 31, the sensor will monitor the current levels over a period of time, reporting the current levels to electronic controller 33. As in the prior embodiments, the controller determines the concentration of the bioagent and displays the result on display 36.

Valve V4 is used to determine the flow speed of fluid through cell 31 by interposing a restrictor R1 and parallel accumulator A1. High flow rates are desirable for flushing the cell after a test. Low rates are better when introducing the charged antibodies so that they are not swept past the electric field by the force of the flow.

As one appreciates, the foregoing describes specific aspects of the mechanization of the ELISA process. The embodiment of FIG. 5 automatically carries out the same functions as earlier described for FIGS. 1, 2 and 3 in automatically accomplishing the ELISA process, which need not be repeated.

The means for holding the sample solution in the prior embodiments was referred to as a vessel. It should be understood that the term vessel in that connection is intended to refer to any region, pipe, conduit or any other suitable means for holding the sample consistent with the described operation, and is to be not limited in meaning to a jar or container and may be referred to as a sample holding means.

The foregoing embodiments were described using antibodies as the recognition molecule for suspect bioagents. As is appreciated it is also desirable on occasion to be able to detect other agents, such as nucleic acid (e.g., DNA) and proteins, a procedure referred to herein as ELISA-like since the ELISA procedures are employed for such detection and the term ELISA may be semantically linked by the medical researchers to be specific to the use of antibodies. For those additional agents the recognition molecule used in the process will likely be a different substance than an antibody. The foregoing description of the embodiments of the invention, however, provides the guide to future researchers to find and isolate appropriate recognition molecules for those additional agents for use in the practice of the present invention.

As earlier described the recognition molecule, as example, may be a monoclonal antibody or, as variously termed, a single-chain variable fragment (“sFv”) molecule. To produce an amino-acid based charged recognition molecule, here referred to as a charged antibody, DNA that codes a string of electrically charged amino acids is spliced onto the coding region of a recognition molecule, such as a monoclonal antibody or single-chain Fv molecule (“sFv”) to produce a chimaeric gene. Amino acids, such as glutamate or aspartate, will produce a negative electric charge, while basic amino acids, such as histidine, will produce a positive electric charge. The particular charge selected depends on the use. The chimaeric gene can be expressed to generate a fusion protein using a number of cell-types as expression systems. As one example a fusion of six glutamates at the carboxyl end of an sFv molecule in a mammalian cell-culture expression system follows.

Generation of sFv molecules from a fragment variable (“Fv”) is described in the literature. See Gilliland LK et al., “Rapid and Reliable Cloning of Antibody Variable Regions and Generation of Recombinant Single Chain Antibody Fragments, “Tissue Antigens, Vol. 47(1), January 1996, pp. 1-20 and Milenic DE et al., “Construction, Binding Properties, Metabolism and Tumor Targeting of a Single-Chain Fv Derived from the Pancarcinoma Monoclonal Antibody CC49,” Cancer Research, Vol. 51(23 Pt 1), December 1991, pp. 6363-71.

A plasmid is a circular piece of DNA that one is able to introduce into cells and constitutes a tool to allow genetic manipulations to be introduced into living cells. The plasmid contains genes whose expression can be driven to produce a protein of interest from a living cell. Beginning with a plasmid that contains an sFv gene of known DNA sequence, two oligonucleotide primers, short pieces of DNA that possesses complementarity to a known portion of a larger region of interest in the DNA sequence, are constructed on the sFv molecule to support a polymerase chain reaction (“PCR”).

The first primer is generally constructed as the codon ATG followed by eighteen nucleotides from the amino-terminal sequence of the sFv molecule. A second primer contains six nucleotides from the carboxyl-terminal sequence of the sFv followed by a number of repeats of the codon GAA equal to the number of glutamates to be added to the molecule, which in this example is six, further followed by two stop codons (e.g., TGA) and a trailing sequence (e.g., GAA GAA GAA GAA GAA GAA TGA TGA GGA GAC GGT GAC CAT GGT).

A PCR reaction is performed and the resultant product is cloned in a single step into pCRII-TOPO, a plasmid sold by the Invitrogen Company of San Diego as a molecular biology research tool. That cloning procedure and product is described in product literature, such as published by Invitrogen company of San Diego. Cloning of monoclonal antibodies is well understood in the art and for additional details the interested reader may refer to Takahashi S et al., “Cloning and cDNA Sequence Analysis of Nephritogenic Monoclonal Antibodies Derived From an MRL/lpr Lupus Mouse, Molecular Immunology,” 1993, Febuary; Vol. 30 (2) pp. 177-182 and Hong HJ et al, “Cloning and Characterization of cDNAs Coding for Heavy and Light Chains of a Monoclonal Antibody Specific for Pre-S2 Antigen of Hepatitis B virus.”

The construct, pCRII-TOPO, is then subcloned into an expression vector, a plasmid that contains a promoter, another stretch of DNA in which transcription factors bind to drive expression of a gene of interest cloned into a specific location and production of the gene's protein product. Subcloning is the process of cutting a stretch of DNA out of a plasmid using restriction enzymes that cut DNA only at specific sequences, referred to as restriction sites, and then cloning that cleaved stretch of DNA into another plasmid.

After the gene is subcloned into an expression vector, the vector is introduced into a cultured cell line (i.e., living cells held in a bathing medium in a petri dish in an incubator). That introduction is accomplished by a chemical introduction process, referred to as transfection, that essentially pokes a hole in the cells that allow the expression vector, a plasmid, to enter through the cell membrane and be trapped inside. The holes are small enough to permit the vector to pass, but not large enough to kill the cell. Just like a “sick” cell that is infected with a virus is hijacked by the virus to produce viral proteins, the transfected cells are hijacked to produce the protein product of the gene introduced by the expression vector. The protein engineered in the foregoing way is engineered to be pushed out of the cells into the bathing medium. In addition to the expressed protein, the bathing medium for the cells also contains the nutrients that sustain the living cell, the cell food, and the waste produced by the cell. The proteins are then extracted from that mixure or “purified.”

One example of a purification process employs a chromatography column, a hollow tube filled with small bead of a porous gel. When a solution containing molecules in a variety of sizes, some larger in size than the pores or holes in the gel and some smaller, is passed through the gel-filled tube, the small molecules can pass into the pores in the gel, while the larger molecules cannot. The smaller molecules will be trapped at least temporarily in the gel, and will flow more slowly through the column than the larger molecules. The result is a separation of the larger molecules based on size. with the larger molecules exiting the tube first. In that way, the desired protein may be separated from the liquid and other contents of the bathing medium.

The preferred purification process is one that takes advantage of the electrical charge, instead of the foregoing mechanical one. The charged molecules are smoothly pumped through a one meter length of tubing at a rate of 10 μl/s. An electrode extends along the length of the tubing and bears a voltage of 0.7 volts, producing an electrostatic field that extends into the tubing. The electrode serves as an electrochemical chromatography column. Charged molecules flowing through the tubing are attracted by the electrostatic field to the oppositely charged electrode and are retained, while other contents in the liquid, such as the cell nutrient and waste product, continue to flow freely through the tubing. The result is a concentration gradient in the solution with more charged molecules at the end of the flow stream. This procedure is repeated on those molecules taken at the end of the flow stream to further purify or enrich the end product with the charged molecules; and repeated again until the charged molecules are sufficiently enriched.

It is believed that the foregoing description of the preferred embodiments of the invention is sufficient in detail to enable one skilled in the art to make and use the invention without undue experimentation. However, it is expressly understood that the detail of the elements comprising the embodiment presented for the foregoing purpose is not intended to limit the scope of the invention in any way, in as much as equivalents to those elements and other modifications thereof, all of which come within the scope of the invention, will become apparent to those skilled in the art upon reading this specification. Thus, the invention is to be broadly construed within the full scope of the appended claims.

1. In a reporter system that detects the presence of a specific biological agent by linking both an electrically charged recognition molecule and a recognition molecule and enzyme complex to the biological agent and introducing a substrate of the enzyme to a liquid containing said biological agent linked electrically charged recognition molecule and recognition molecule and enzyme complex, wherein the enzyme cleaves the substrate to release a reporter in said liquid, said reporter system including a sensor for sensing the amount of said reporter in said liquid, the improvement comprising:
electric field generating means for generating an electric field in the vicinity of said sensor to attract said biological agent and linked electrically charged recognition molecule and recognition molecule and enzyme complex to the vicinity of said sensor.

2. The method of detecting a particular agent that comprises a bioagent, protein, or nucleic acid, comprising the steps of:
mixing said agent in a solution containing an electrically charged recognition molecule to said agent to form a 1° recognition molecule/agent complex;
mixing a 2° recognition molecule enzyme complex together with said 1° recognition molecule/agent complex to form a 1° agent/recognition molecule/2° recognition molecule enzyme complex;
applying an electric field to said 1° agent/recognition molecule/2° recognition molecule enzyme complex for positioning said 1° agent/recognition molecule/2° recognition molecule enzyme complex at a reporter sensing location;
introducing a substrate of said enzyme to said 1° agent/recognition molecule/2° recognition molecule enzyme complex to cleave a reporter from said substrate at said reporter sensing location; and
sensing said reporter.

3. The method of detecting a particular agent that comprises a bioagent, protein, or nucleic acid, as defined in claim 2, which includes, prior to said step of mixing said agent in a solution containing an electrically charged recognition molecule to said agent to form a 1° recognition molecule/agent complex, the step of imparting an electrical charge to said recognition molecule.

4. The method of detecting a particular agent that comprises a bioagent, protein, or nucleic acid, as defined in claim 3, wherein said step of imparting an electrical charge to said recognition molecule, includes the step of molecularly splicing a polyglutamate to said recognition molecule.

5. Apparatus for conducting an electrochemical enzyme linked immunosorbent assay (“ELISA”) for a bioagent, protein or nucleic acid comprising:
a plurality of vessels, said plurality of vessels including:
a first vessel for holding electrically charged recognition molecules in liquid;
a second vessel for holding a wash solution;
a third vessel for holding a 2° recognition molecule linked enzyme;
a fourth vessel for holding a substrate reporter; and
sample holding means for holding a sample solution containing said bioagent, protein or nucleic acid;
an examination cell;
an electronic controller, said electronic controller including a program, a start switch and a display;
a sensor for electrically detecting the level of reporter present in said examination cell at any moment of time and supplying said detected level of reporter present at any moment in time to said electronic controller;
an electric field generator controlled by said electronic controller for producing an electric field inside said examination cell when required by said program;
said electronic controller for motivating passage of the respective contents of each of said vessels into said examination cell when required by said program and for motivating removal of the contents of said examination cell in whole and/or in part when required by said program;
said program further defining an ELISA, wherein motivation of each of said vessels is motivated in a sequence to pass contents of the respective vessel into said examination cell to perform an ELISA;
said program defining said ELISA including means for motivating the contents of said fourth vessel into said examination cell and initiating assembly of the detected level of reporter present at each of a plurality of time intervals from said sensor; whereby insertion of the contents of said fourth vessel into said examination cell when said bioagent is present in said examination cell produces an electrochemical redox recycling reaction inside said examination cell to produce levels of reporter that increases with time;
wherein said ELISA includes means for washing the fluid in said examination cell;
said means for washing including:
first means for motivating said electric field generator to produce an electric field in said examination cell responsive to a command from said electronic controller, wherein said electrically charged recognition molecules in said examination cell are drawn to one side of said examination cell leaving a portion of said examination cell free of recognition molecules;
second means for aspirating fluid from said region of said examination cell vacated by said recognition molecules while said electric field is present responsive to a command from said electronic controller, and
third means for motivating wash fluid from said second vessel into said examination cell responsive to a command by said electronic controller following aspiration of fluid by said second means;
said program further including an analysis program for analyzing the detected level of reporter at each of said plurality of time intervals and determining the concentration of bioagent present in said sample when said bioagent is present in said sample and displaying said concentration on said display.

6. The apparatus for conducting an electrochemical ELISA for a bioagent, protein or nucleic acid as defined in claim 5, wherein said means for motivating the contents of said fourth vessel into said examination cell and initiating assembly of the detected level of reporter present at each of a plurality of time intervals from said sensor further includes:
means for motivating said electric field generator to produce an electric field in said examination cell that extends through said sensor, wherein said electrically charged recognition molecules in said examination tube are drawn to said sensor.

7. Apparatus for conducting an electrochemical enzyme linked immunosorbent assay (“ELISA”) for a bioagent, protein or nucleic acid comprising:
a plurality of vessels, said plurality of vessels including:
a first vessel for holding 1° antibodies in liquid;
a second vessel for holding a wash solution;
a third vessel for holding a 2° antibody linked enzyme;
a fourth vessel for holding a substrate reporter; and
sample holding means for holding a sample solution containing said bioagent;
an examination cell;
a plurality of electric pumps, each pump being associated with a respective one of said first through fourth vessels and said sample holding means for conveying contents from the respective vessel into said examination cell when said pump is energized;
an electronic controller, said electronic controller including a program, a start switch and a display;
said electronic controller being coupled to said plurality of pumps for controlling the energization of said pumps in accordance with said controller program;
said electronic controller further including a look-up table, said look-up table containing a plurality of numbers defining slopes and a plurality of bioagent concentrations with each of said plurality of bioagent concentrations being associated with a respective one of said plurality of numbers, wherein for each slope represented in said look-up table, a concentration of said bioagent may be determined;
an aspirating pump, said aspirating pump for pumping contents from said examination cell when energized by said electronic controller;
an electric field generator controlled by said electronic controller for producing an electric field inside said examination cell when required by said program;
a sensor for electrically detecting the level of reporter present in said examination cell at any moment of time and supplying said detected level of reporter present at any moment in time to said electronic controller, said sensor for producing an electrical current that is proportional to the quantity of reporter present in said examination cell, whereby the electrical current level increases as the amount of reporter developed with time in said examination cell increases;
said sensor further comprising a pair of spaced comb-like shaped electrodes interdigitally arranged, said spaced electrodes being located inside said examination cell to provide a current carrying path in a gap between said electrodes through at least a portion of the contents of said examination cell, and a pair of electrical conductors for respectively connecting each of said spaced electrodes to a source of potential external to said examination cell;
said electronic controller for motivating passage of the respective contents of each of said vessels into said examination cell when required by said program and for motivating removal of the contents of said examination cell in whole and/or in part when required by said program;
said program further defining an ELISA, wherein motivation of each of said vessels is motivated in a sequence to pass contents of the respective vessel into said examination cell to perform an ELISA;
said ELISA including means for washing the fluid in said examination cell, said means for washing further comprising:
first means for motivating said electric field generator to produce an electric field in said examination cell responsive to a command from said electronic controller, wherein electrically charged recognition molecules in said examination cell are drawn toward a side of said examination cell leaving a portion of said examination cell vacant of recognition molecules;
second means for aspirating fluid from said bead vacated region of said examination cell while said electric field is present responsive to a command from said electronic controller, and
third means for motivating wash fluid from said second vessel into said examination cell responsive to a command by said electronic controller following aspiration of fluid by said second means;
said program defining said ELISA including means for motivating the contents of said fourth vessel into said examination cell, motivating said electric field generator to produce an electric field in said examination cell that extends through said sensor to draw electrically charged recognition molecules in said examination tube to said sensor, and initiating assembly of the detected level of reporter present at each of a plurality of time intervals from said sensor; whereby insertion of the contents of said fourth vessel into said examination cell when said bioagent is present in said examination cell produces an electrochemical reaction inside said examination cell to produce levels of reporter that increases with time;
said program further including an analysis program for analyzing the detected level of reporter at each of said plurality of time intervals and determining the concentration of bioagent present in said sample when said bioagent is present in said sample and displaying said concentration on said display;
said analysis program including:
a regression analysis program for performing a least-square linear regression analysis on said detected level of reporter taken at each of said plurality of time intervals to determine a number, said number defining a slope;
a look up program for looking up said number determined by said regression analysis program in said look-up table and locating the corresponding concentration of said bioagent represented thereby.

8. The method of fabricating an electrically charged recognition molecule comprising the steps of:
molecularly splicing a string of DNA that codes for a polyglutamate into a proteinaceous recognition molecule of known DNA sequence to produce a combined DNA sequence, said recognition molecule being composed of amino acids; and
inserting said combined DNA sequence into a producer cell, wherein said producer cell produces electrically charged recognition molecules.
